(19) United States
(12) Patent Application Publication
(10) Pub. No.: US 2018/0368750 A1
(43) Pub. Date: Dec. 27, 2018

(54) METHOD FOR DETERMINING THE INDIVIDUAL BITING FORCE OF A PATIENT

(71) Applicant: SICAT GMBH & CO. KG, BONN (DE)

(72) Inventors: NILS HANSSEN, BONN (DE); JOACHIM HEY, KOENIGSWINTER (DE); JOCHEN KUSCH, WACHTBERG (DE); TOBIAS LEHNER, BONN (DE)

(73) Assignee: SICAT GMBH & CO. KG, BONN (DE)

(21) Appl. No.: 15/779,544

(22) PCT Filed: Nov. 25, 2016
(86) PCT No.: PCT/EP2016/078764

§ 371 Date: May 29, 2018

(30) Foreign Application Priority Data
Nov. 30, 2015 (DE) 10 2015 120 744.3

(51) Int. Cl. A61B 5/22; A61C 19/05

(52) U.S. Cl. CPC A61B 5/228; A61C 19/05

(57) ABSTRACT

A method for determining an individual biting force of a patient includes providing a test piece with a deformable nature, individually pre-shaping a surface of the test piece for the patient so as to obtain at least one of a defined positioning of the test piece on teeth of the patient and on a device supported by at least one of an upper jaw and a lower jaw of the patient, introducing the test piece between the upper jaw and the lower jaw of the patient, biting, via the patient, onto the test piece so as to provide a deformation of the test piece, and determining the individual biting force by examining the deformation of the test piece.

### CROSS REFERENCE TO PRIOR APPLICATIONS

This application is a U.S. National Phase application under 35 U.S.C. § 371 of International Application No. PCT/EP2016/078764, filed on Nov. 25, 2016 and which claims benefit to German Patent Application No. 10 2015 120 744.3, filed on Nov. 30, 2015. The International Application was published in German on Jun. 8, 2017 as WO 2017/093128 A1 under PCT Article 21(2).

### FIELD

The present invention relates to a method and a test piece for determining the individual biting force of a patient by biting onto a test piece of a deformable nature introduced between upper jaw and lower jaw, wherein the teeth of the patient and/or device supported by the jaw, for example a crown, a bridge or a dental splint, press into a surface of the test piece during biting, and wherein the biting force is determined by examining the deformation of material caused by the biting process.

### BACKGROUND

Such measurements of biting forces are generally of great importance for therapeutic purposes. It is, for example, advantageous to consider the distribution of forces that occur during a bite when planning implants. From such biting force tests, the causes of pain in the jaw region can be also determined and accordingly treated in a targeted manner. Force measurements are of interest when planning therapeutic splints in order to minimize and compensate for the biting forces of the patient; load peaks occurring at points should be deliberately avoided by therapeutic splints.

Simple methods have previously been described to determine the biting force which use the change in electrical resistance or capacitance under the effect of pressure. The use of deformation-sensitive piezoelectric films has also previously been described. A particularly simple method makes use of a horseshoe-shaped bite foil with a pressure-sensitive film.

DE 10 2013 211 623 A1 describes a method for determining the biting force in which method the patient bites onto a test piece of elastic and/or deformable material placed between the teeth and so as to press into the smooth surface of the test piece. The deformation of the material is recorded electronically by a motion detection system which detects the jaw movements, with the biting force acting on the material being calculated from the deformation based on the material properties of the deformable material, for example, via the finite elements method (FEM). To be able to calculate the deformation of the material at any time during the measurement of the mastication movement, DE 10 2013 211 623 A1 uses digital tooth impressions which are in the correct spatial relationship to the recorded motion data. The compression of the material by the patient's teeth can thereby be determined at any time, and the resulting forces can be determined.

The method described in DE 10 2013 211 623 A1 only makes it possible to determine the mean values of the forces for one defined tooth quadrant at a time, since contact surfaces between teeth and material are not defined. A further disadvantage is that the position of the test piece with respect to the teeth at the time of measurement is arbitrary, and thus unknown. Without knowing the exact position of the test piece, however, the simulation by FEM and therefore the calculation of the biting force can only be carried out inexactly, if at all. The position of the test piece in this case must be determined by a further measurement, for example, by optical scanning of the elastic material in the measurement position.

U.S. Pat. No. 4,488,873 describes a method for determining the biting force of an individual patient in which method the surface of a test piece is individually shaped by the teeth biting thereon.

### SUMMARY

An aspect of the present invention is to provide a method which can be simply implemented, and a corresponding test piece via which the biting forces of individual patients can be better determined with spatial resolution.

In an embodiment, the present invention provides a method for determining an individual biting force of a patient which includes providing a test piece comprising a deformable nature, individually pre-shaping a surface of the test piece for the patient so as to obtain at least one of a defined positioning of the test piece on teeth of the patient and on a device supported by at least one of an upper jaw and a lower jaw of the patient, introducing the test piece between the upper jaw and the lower jaw of the patient, biting, via the patient, onto the test piece so as to provide a deformation of the test piece, and determining the individual biting force by examining the deformation of the test piece.

### BRIEF DESCRIPTION OF THE DRAWINGS

The present invention is described in greater detail below on the basis of embodiments and of the drawings in which:

FIG. 1 shows a side view of a set of human teeth, with a test piece made of printed rubber bearing on both sides;

FIG. 2 shows a strip-shaped test piece bearing on the upper jaw;

FIG. 3 shows a strip-shaped test piece bearing on the lower jaw;

FIG. 4 shows two strip-shaped test pieces;

FIG. 5 shows an intermediate layer introduced between the test pieces according to FIG. 4;

FIG. 6 shows a sensor foil additionally introduced between strip-shaped test pieces;

FIG. 7 shows a test piece made of printed rubber in a defined opening position, which test piece bears on the lower jaw with at least partial form-fit engagement and, on the upper jaw, replicates the contact points of the teeth of the lower jaw;

FIG. 8 shows a printed tooth inserted into a main body; and

FIG. 9 shows a front view of dentition with a dental splint.

### DETAILED DESCRIPTION

A core aspect of the present invention is that the elastic material of the test piece is provided from the outset with a defined shape individually adapted to the conditions between the jaws, and that this exact fit makes it possible to define the position between the jaws of the patient to be tested. A surface of the test piece is thereby shaped individually for the patient prior to biting so that the teeth acting on the test piece and/or the corresponding device supported by the jaw have a defined bearing position during biting.

A test piece according to the present invention can be made in one piece from a pre-shaped elastic material or can have a main body which is covered with pre-shaped surfaces of elastic material. The main body itself can be rigid or elastic. The elastic material does not need to have a homogeneous elasticity.

It is advantageous if the elasticity of the material in the front jaw regions is different than in the rear jaw regions due to the rotatory opening of the jaws. Depending on the application, it may also be advantageous to use a visco-elastic material that allows the patient to bite almost as far as the terminal occlusion.

The elastic material of the test piece is ideally pre-shaped so that the anatomical or implant conditions, i.e., the tooth surfaces and/or implant surfaces, are embedded at least on one side in the elastic material of the test piece, resulting in a defined planar bearing in the deformable elastic material. If biting is now performed with a defined biting force, the pre-shaped material yields uniformly everywhere, in particular with a homogeneously distributed counterforce. The predefined deformation is uniformly impressed by the bite and not first generated as in the prior art. Individual tooth cusps can now be loaded or unloaded in a targeted manner via a specific configuration of the pre-shaped material.

To express this visually, the approach according to the present invention corresponds to a crash test in which the deformation after the force impact is examined and, with knowledge of the material properties of the deformed material, in the present case the test piece with the flexible surface, the force impact is determined in particular by FEM. According to the present invention, however, the test piece is pre-shaped corresponding to the surface of the formation that is acted on by force. The test piece according to the present invention can moreover be constructed heterogeneously from different elastic materials.

The surface is advantageously pre-shaped by rapid prototyping or 3D printing. Many elastic materials, such as rubber or silicone, can be brought into any desired shape with such methods. It is even possible with 3D printing to produce a monolithic workpiece which has different degrees of hardness at different locations. It is thereby possible to produce test pieces optimally adapted to existing conditions. The test pieces can be prepared on the basis of previously recorded three-dimensional image data.

The pre-shaping of the elastic material according to the present invention affords various advantages:

Through the at least one-sided form-fit engagement, the position of the deformable material in relation to the teeth is exactly defined. An optical scan to determine the position of the elastic material is therefore no longer necessary. Because of the precise fit, the test piece can be inserted between the jaws only in the desired position. A correspondingly precise force calculation can be performed on the basis of the exact position of the elastic material. It suffices here if the test piece on one side of the jaw bears with form-fit engagement only at a small number of defined locations.

The forces acting on a tooth relief can also be taken into consideration in advance, for example, if an individual cusp (antagonist form or the like) is present. The shape of the elastic material can correspondingly be configured for the force measurement.

The test piece can moreover be optimized to the load situation through use of different Shore hardnesses. It can also be designed so that the jaw must apply an increasing closure force as the bite closes. The directions of the vectors are known from motion data.

By predefining the shape of the deformable material, it can also be achieved that the patient bites on the material with a predetermined jaw position and a defined jaw orientation.

Finally, the position of any restorations can be exactly predefined by the form-fit or force-fit engagements for rubber.

With the present invention, it is possible, for example, to measure the force distribution on a therapeutic splint in order to compensate for a malpositioning of the jaw. The patient in this case wears a system for measuring the jaw movement. By measuring the jaw movements, the movements can be transferred to the tooth impressions existing as digital data (virtual articulator). A therapeutic splint configured according to the present invention and made of pre-shaped rubber is thereby present in the patient's mouth. The patient then performs various closure movements of the jaws. The compression of the splint can be determined on the basis of the digital impressions with the knowledge of the precise position of the pre-shaped therapeutic splint according to the present invention. The forces existing between the teeth and the rubber splint, and caused by the measured excursions, are calculated by FEM simulation. The more the forces on the therapeutic splint are uniformly distributed, the better the therapeutic outcome will be.

In another use, the force distribution of a natural terminal occlusion can be measured. For this purpose, the patient again wears a system for measuring the jaw movement. Through the measurement of the jaw movements, it is again possible to transfer the movements to the impressions existing in digital form. Between his/her teeth, the patient wears a sufficiently thick, pre-shaped rubber according to the present invention, which bears at least partially with form-fit engagement on the teeth of the lower jaw and, on the opposite side, replicates the geometry of the teeth of the lower jaw. If the patient now bites his/her teeth together, the same points between the (rubber) teeth of the lower jaw and the teeth of the upper jaw have contact as in the natural occlusion. Various jaw closure movements are performed. Since the geometry and position of the rubber are known, it is possible to determine the compression of the rubber and, therefrom, the forces at the contact points between the teeth of the upper jaw and the (rubber) teeth of the lower jaw. A treatment of the patient with a therapeutic splint is recommended if the forces are confirmed to be distributed unevenly (also over time).

With the approach according to the present invention, the force distribution can also be determined on dental splints, such as occlusion splints in the patient's mouth. Such splints are used, for example, to compensate the occlusion bite contacts and to uniformly distribute the forces of the contact points. According to the present invention, a planned prototype splint is printed from rubber and introduced into the patient's mouth therefor. The forces on the planned splint during biting are determined by measuring the movement with the rubber splint. The softer the splint, the more quickly the patient achieves an equilibrium of the biting forces. Since the forces are calculated through the simulation, a criterion can be defined in which the forces are compensated. The position in which the teeth are at sufficient equilibrium can be recorded and utilized for the production of an optimized splint. The printed prototype splint need not be made completely of an elastic material, and can instead be a combination of soft and hard material. The form-fit part can be soft while the part contacting the opposite teeth can be hard. The patient can thereby perform sliding movements with the prototype splint as with the final splint, and transverse forces can be measured with the aid of the rubber layer.

Various design variants are now possible with regard to the configuration of the test piece. For example, in a simple design variant, the flexible material of the test piece can bear with form-fit engagement either only on the upper jaw or only on the lower jaw. The respective other side can have any desired shape and can in particular be plane. In a more complex embodiment, form-fit engagement can be provided both on the upper jaw and on the lower jaw. In this embodiment, a defined position of the lower jaw and/or angle of the lower jaw can be specifically predefined.

The present invention is described in more detail below under reference to the drawings.

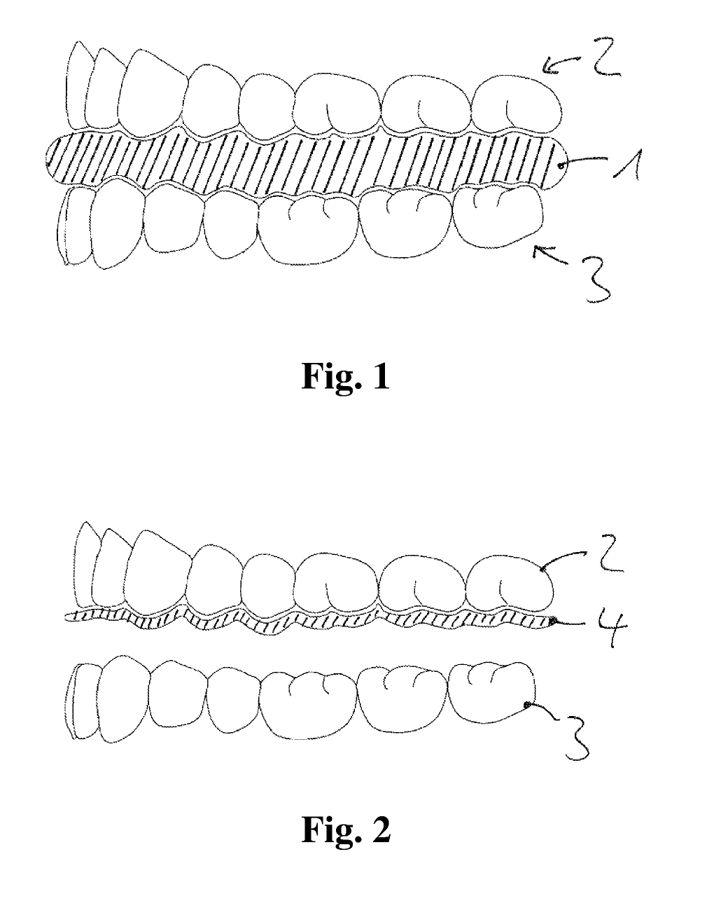

FIG. 1 shows a side view of a set of human teeth. The test piece 1 is a piece of rubber which has been pre-shaped by a 3D printing process and which bears with a form-fit and/or a force-fit engagement both on the teeth 2 of the upper jaw and also on the teeth 3 of the lower jaw. The patient has yet to apply a biting force in the position shown.

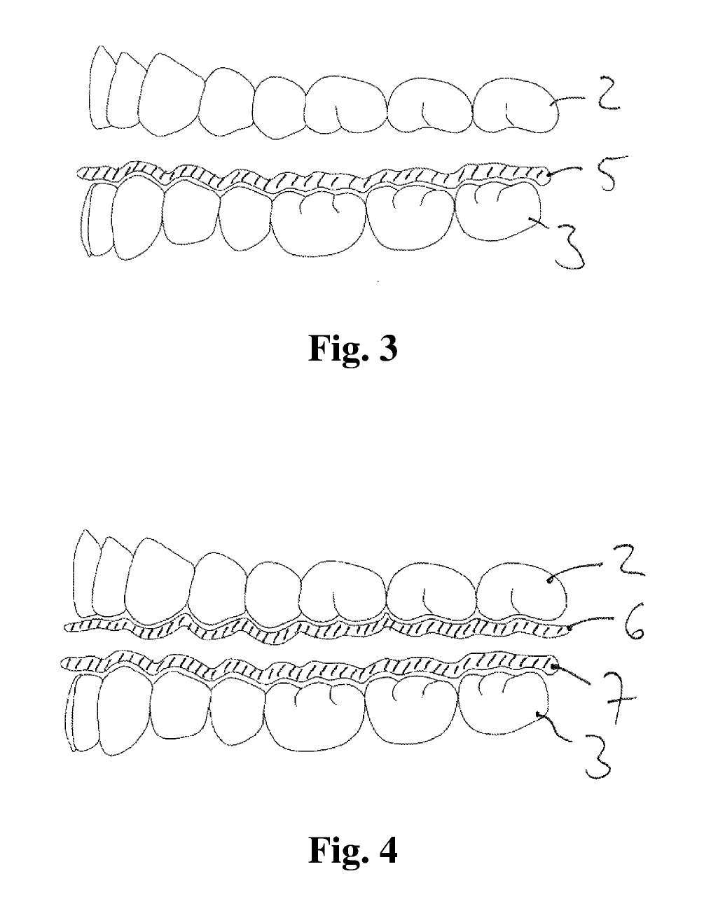

In the variant according to FIG. 2, the test piece 4 is a thin strip of printed rubber bearing on the teeth 2 of the upper jaw. The test piece 4 replicates the shape of the upper teeth 2 in this case. The loading situation of the terminal occlusion can be simulated with the actual tooth contacts with such a test piece (test strip). This also applies in the variant according to FIG. 3 in which the test piece 5 bears on the teeth 3 of the lower jaw. The surface of the respective test strips that faces away from the teeth has a structure corresponding to the opposite warpage, thereby replicating the surface of the opposite teeth. Particularly in the case of small openings of the jaw, this can be achieved by the fact that the test piece 4 is integrally formed as a rubber strip of uniform thickness onto the teeth.

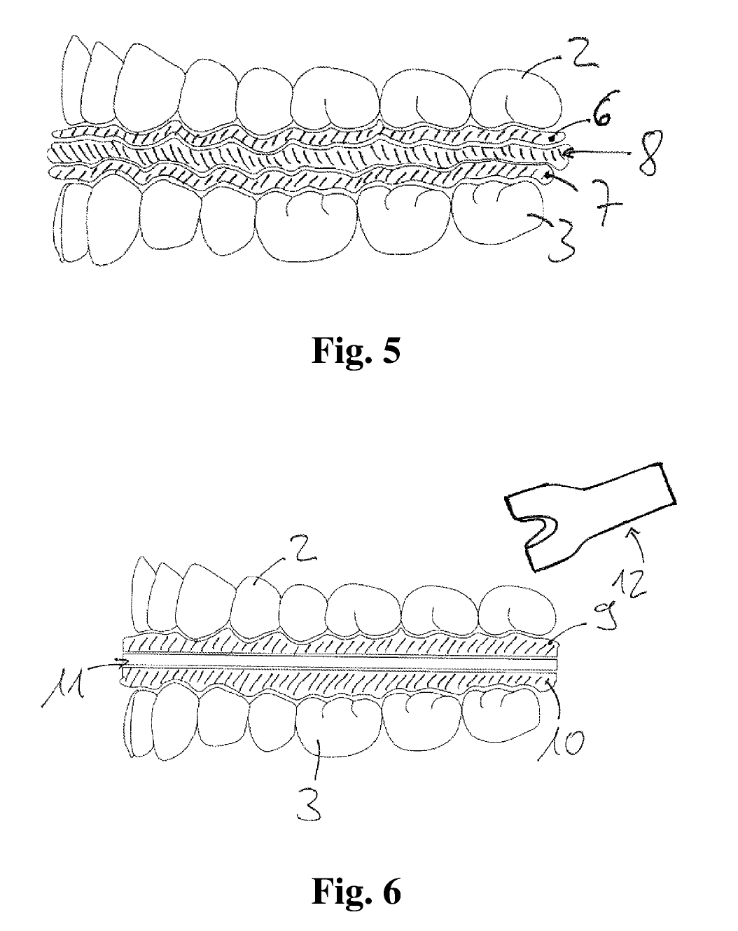

In the variant according to FIG. 4, a test piece 6 in the form of a layer of printed rubber bears with form-fit engagement on the teeth 2 of the upper jaw, and a test piece 7 bears on the teeth 3 of the lower jaw. This variant is the combination of the two aforementioned ones, whereby, in this case, the teeth of both rows of teeth are simulated in the terminal occlusion. In the variant according to FIG. 5, a further central additional layer 8 of defined thickness and of individual configuration is now introduced between the two test strips of printed rubber which, in accordance with the variant according to FIG. 4, bear on the rows of teeth of both jaws. With several additional layers, or with additional layers of different thickness, the biting forces can be determined in different jaw closures and in different dynamic ranges. The central additional layer 8 can be a layer which is available in different thicknesses, hardnesses and jaw angles, and which can accordingly be exchanged. The central additional layer 8 can also have regions of different elasticity.

The variant according to FIG. 6 corresponds to that of FIG. 5, except that the prosthesis strips 9 and 10 of printed rubber each have a plane surface facing away from the teeth. This has the advantage that only the upper and the lower rubber element must be prepared individually for the patient. The central additional layer 11 is universal and can be used and reused for different patients. The central additional layer 11 can in turn be stocked in different hardnesses, thicknesses and jaw angles and can be rapidly exchanged between individual measurement steps.

A commercially available sensor foil 12 can also be placed between the plane boundary faces of the prosthesis test strips 9 and 10. Additional information concerning the biting force can thereby be determined and can be taken into consideration in the FEM simulation. By virtue of the plane boundary surfaces, the measurement is not distorted, as it is in a normal foil measurement, at the location where the foil is warped by the tooth fissures. A sensor foil 12 introduced between the planar boundary surfaces can supplement the FEM force calculation and/or can be used for the absolute calibration of the biting forces.

As set forth above, the elastic material need not necessarily be of such a nature that it recovers its original shape upon removal of a load. It can also be a visco-elastic material which continues to deform without the opposing force of the material further increasing. The patient is thereby able to bite as far as the individual terminal occlusion so that a squeeze bite register is obtained and a force measurement is possible as far as the terminal occlusion. The resulting forces can be calculated as far as the terminal occlusion situation with knowledge of the in particular visco-elastic material properties. Optical capture of the material thus deformed all the way to the terminal occlusion can provide further information concerning the deformation path, if the latter cannot be determined completely by the simulation.

As regards the dimensioning of the test piece, it must be noted that the thicker the deformable material located between the teeth, the better the forces can be calculated, since the deformation of the material can be measured over a greater number of path quantizations. On the other hand, the teeth move apart at an angle to each other with larger openings, i.e., the front teeth typically have a greater distance than the back teeth. The lower jaw also moves forward the further the mouth is opened since the tempero-mandicular joint is a hinging/sliding joint. The angle ratio and the advance of the lower jaw are individual to a patient.

In the illustrative embodiment below, the test piece made of pre-shaped rubber is dimensioned so that it matches the opening angle ratio and the forward thrust of the lower jaw of the individual patient.

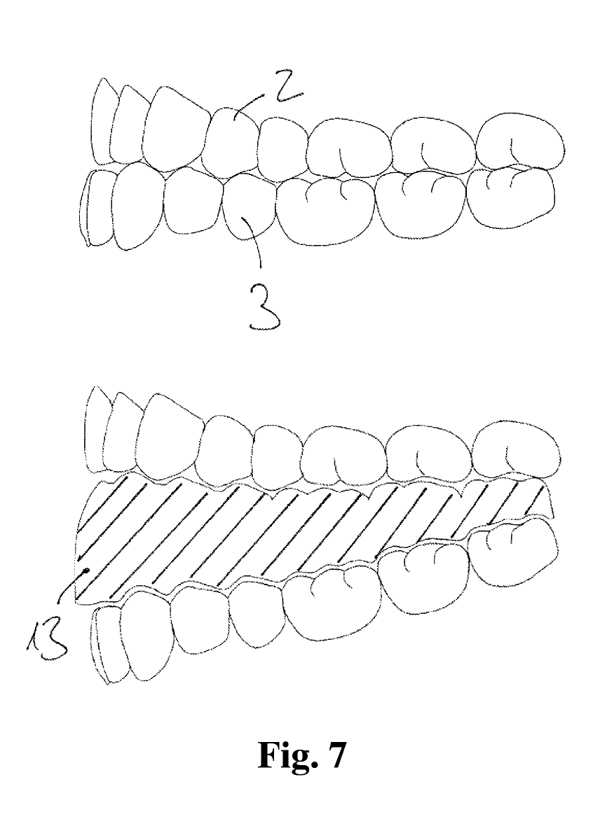

FIG. 7, top, shows the teeth in the natural occlusion bite. The teeth 2 of the upper jaw here have defined contact points, individual to the patient, on the teeth 3 of the lower jaw. In a test piece pre-shaped in this state, the contact points would not be the same as in the natural terminal occlusion. FIG. 7, bottom, shows a wedge-like test piece 13 of printed rubber adapted to the individual opening angle of the patient. The underside of the wedge-like test piece 13 bears with form-fit engagement on the teeth 3 of the lower jaw, the upper face of the rubber replicates the shape of the teeth 2 of the lower jaw as in the natural terminal occlusion and also compensates for the forward thrust of the lower jaw in the case of large opening.

In this embodiment, a force measurement can now be performed with the same contact points as in the natural terminal occlusion. Although the jaw muscles work at slightly different angles compared to the genuine terminal occlusion, this error information can also be calculated in the simulation based on the knowledge of the opening angle. The Shore hardness of the rubber at the front teeth can also be chosen to be less than at the molars. A linearized force response can be generated via a suitable distribution of the Shore hardnesses since the front teeth travel a greater path than the molars. The reverse scenario can also be set, wherein the rubber on the upper face bears with form-fit/force-fit engagement on the upper jaw and the underside of the rubber replicates the shape of the teeth of the upper jaw. The lower jaw variant has the advantage, however, that the rubber can bear on the teeth and need not be secured with force-fit engagement on the teeth of the upper jaw.

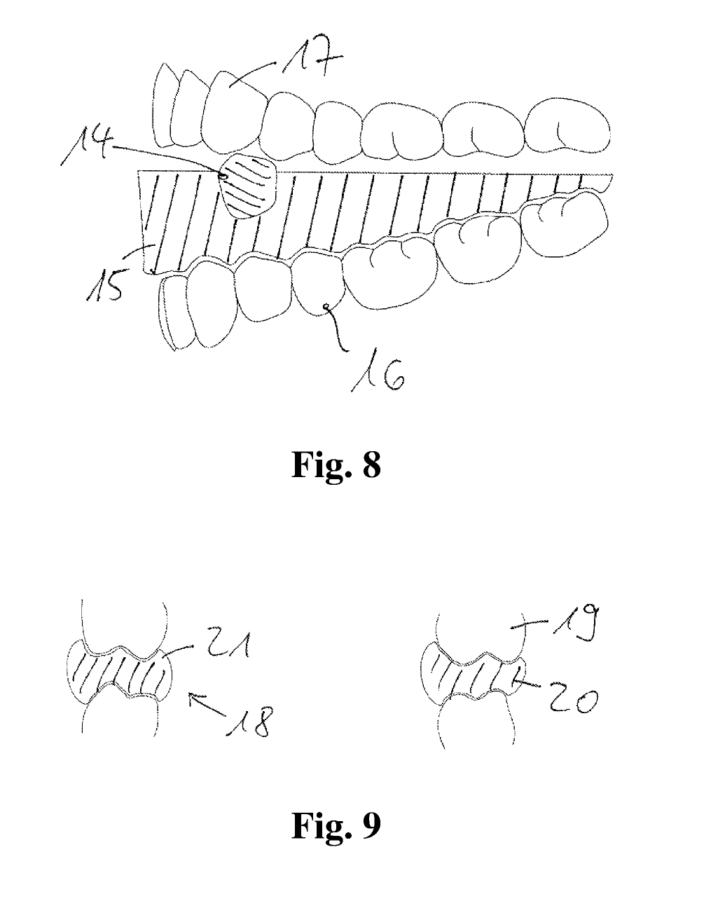

FIG. 8 shows a situation similar to that of FIG. 7. However, a single printed tooth 14 is here inserted into the wedge-like pre-shaped test piece 15. The printed tooth 14 corresponds to the natural tooth 16 lying below it or replaces a gap that is intended to be closed by a crown. The terminal occlusion load on certain teeth can thereby be limited. The remaining teeth of the row 17 of teeth have no contact during the measurement. The inserted printed tooth 14 can, as here, correspond to the natural shape of an existing tooth or also to a planned crown. The printed tooth 14 can be made of rubber or a harder material since the wedge-like rubber blank yields and is thus suitable for the force measurement. The wedge 15 can also be made of a hard material, with only the inserted printed tooth 14 being made of rubber. The same tooth can, however, be inserted into wedges of different levels of rubber hardness. Via mechanical recesses provided all around the wedge-like pre-shaped test piece 15, different individual teeth or groups of teeth can be inserted in the wedge-like pre-shaped test piece 15 at each tooth position.

FIG. 9 shows a frontal section through the molars, looking from the front into the patient's mouth. The dental splint bears with form-fit engagement on the right-hand side 18 of the jaw and on the left-hand side 19 of the jaw, at least on the upper jaw or lower jaw. The rubber material 20 on the left-hand side is more elastic than the rubber material 21 on the right-hand side. The patient can thereby be brought more quickly to an equilibrium of the forces on one side. Different elasticities could be obtained not only by using different base materials, but also by printing cavities or microstructures which are able to control the elasticity of different regions of the printed product in a targeted manner. The cavities could also be filled with a liquid so that various regions of the printed product “communicate” with one another (communicating ducts) and thus establish an equilibrium more quickly.

Via a form-fit configuration on both jaws, it is possible to determine not only closure forces, but also lateral and/or protruding and/or retruding forces. Opening forces could also be determined in a force-fit configuration.

The present invention is not limited to embodiments described herein; reference should be had to the appended claims.

What is claimed is:

1-10. (canceled)

11. A method for determining an individual biting force of a patient, the method comprising:
providing a test piece comprising a deformable nature;
individually pre-shaping a surface of the test piece for the patient so as to obtain at least one of a defined positioning of the test piece on teeth of the patient and on a device supported by at least one of an upper jaw and a lower jaw of the patient;
introducing the test piece between the upper jaw and the lower jaw of the patient;
biting, via the patient, onto the test piece so as to provide a deformation of the test piece; and
determining the individual biting force by examining the deformation of the test piece.

12. The method as recited in claim 11, wherein the individual pre-shaping of the surface of the test piece for the patient is performed using an impression of acting teeth.

13. The method as recited in claim 11, wherein,
the deformable nature of the test piece is provided by a deformable material, and
the test piece is produced completely or partially from the deformable material.

14. The method as recited in claim 13, wherein the deformable material is individually pre-shaped by a rapid prototyping method so as to provide a pre-shaped deformable material.

15. The method as recited in claim 14, wherein the pre-shaped deformable material bears with a form-fit engagement on teeth of the upper jaw and on teeth of the lower jaw so as to predefine a specific jaw position for a force measurement.

16. The method as recited in claim 14, wherein the pre-shaped deformable material is configured to replicate a normal occlusion or another reference position with a different vertical blocking in another vertical blocking.

17. The method as recited in claim 11, wherein the individual biting force is determined by examining an impression created by the bite and by simulating the impression via a finite elements method (FEM).

18. A test piece for use in the method as recited in claim 11, the test piece comprising:
a deformable nature; and
a pre-shaped surface which is shaped individually for a patient and which bears in a defined manner on at least one of teeth of the patient and a device supported by at least one of an upper jaw and a lower jaw of the patient.

19. The test piece as recited in claim 18, wherein,
the test piece comprises only one pre-shaped surface which bears with a form-fit engagement on teeth either of the upper jaw or of the lower jaw of the patient, and
the other surface replicates bearing teeth of the patient.

20. The test piece as recited in claim 18, wherein,
the test piece comprises a first pre-shaped surface and a second pre-shaped surface,
the teeth of the upper jaw of the patient bear with a form-fit engagement on the first pre-shaped surface, and
the teeth of the lower jaw of the patient bear with a form-fit engagement on the second pre-shaped surface.
